(19) United States
(12) Patent Application Publication
(10) Pub. No.: US 2008/0139898 A1
(43) Pub. Date: Jun. 12, 2008

(54) System and Method For Providing Centralized Physiological Monitoring

(76) Inventors: Timothy Lee Johnson, Niskayuna, NY (US); Xi Wang, Albany, NY (US); Timothy Joseph Kottak, Pewaukee, WI (US); Stephen Treacy, Milwaukee, WI (US)

Correspondence Address: BEUSSE WOLTER SANKS MORA & MAIRE, P. A., 390 NORTH ORANGE AVENUE, SUITE 2500, ORLANDO, FL, 32801, US

(21) Appl. No.: 11/567,758

(22) Filed: Dec. 7, 2006

(51) Int. Cl. A61B 5/00

(52) U.S. Cl. 600/301

(57) ABSTRACT

A system and method for reducing a workload of a user monitoring respective physiological conditions of a plurality of patients at a centralized physiological monitoring station. The system includes a plurality of patient measurement systems generating respective alarms indicative of a patient's physiological condition and a centralized physiological monitoring station receiving the alarms from the patient measurement systems. A processor in communication with the centralized physiological monitoring station is configured to monitor patterns in the alarms for each of the patients, to establish individualized historical alarm information for each of the patients responsive to monitored patterns in the alarms, to determine an urgency of the respective alarms according to the individualized historical alarm information, and to provide notifications to a user monitoring the respective physiological conditions at the centralized physiological monitoring station. The system also includes a display in communication with the processor for displaying the notifications to the user.

### FIELD OF THE INVENTION

Embodiments of the present invention are generally related to centralized physiological monitoring of patients, and, more particularly, to a system and method for reducing the workload of a tele-tech monitoring physiological conditions of a plurality of patients at a centralized physiological monitoring station.

### BACKGROUND OF THE INVENTION

Various devices are known for monitoring physiological parameters, such as electrocardiogram (ECG), non-invasive blood pressure (NBP), and specific blood oxygen (SpO2) of patients being treated for medical conditions. Typically, such devices continually provide vital sign information based on the monitored physiological parameters to enable medical personnel, such as physicians, nurses, and other health care providers to provide appropriate care. However, in typical hospital environments where numerous patients are cared for, it can be difficult for medical personnel to monitor the vital sings of multiple patients on a continuous basis while maintaining responsiveness to problems of an individual patient.

In an attempt to alleviate demands on medical personnel for monitoring vital signs of multiple patients, centralized monitoring systems that collect physiological data from monitored patients and display the collected data at a central location have been developed. Such centralized monitoring systems are typically manned by telemetry technicians, or “tele-techs”, to alleviate a workload of medical personnel in providing direct care to patients. Vital sign information provided to the tele-tech is typically in the form of alerts or alarms. Upon recognizing an alert or alarm condition, the tele-tech notifies a clinician or nurse assigned to provide care to the corresponding patient generating the alert or alarm. Although such systems shift a continuous monitoring burden away from clinicians, tele-techs monitoring vital signs at a centralized location may experience work overload when they are required to monitor and respond to too many alerts and alarms within a prohibitively short period of time.

### BRIEF DESCRIPTION OF THE INVENTION

In an example embodiment, the invention includes a system for reducing a workload of a user monitoring respective physiological conditions of a plurality of patients at a centralized physiological monitoring station. The system includes a plurality of patient measurement systems generating respective alarms indicative of a patient's physiological condition and a centralized physiological monitoring station receiving the alarms from the patient measurement systems. The system also includes a processing module in communication with the centralized physiological monitoring station configured for adaptively controlling a presentation of the alarms at the centralized physiological monitoring station responsive to at least one of patient history information, historical alarm information, electro-physiological data, quality of service data, and staff assignment information so as to reduce a workload on a user monitoring the presentation of the alarms and a display in communication with the adaptive processing module for displaying the presentation of the alarms to the user at the centralized physiological monitoring station.

In another example embodiment, the invention includes a method for reducing a workload of a user monitoring respective physiological conditions of a plurality of patients at a centralized physiological monitoring station. The method includes providing a plurality of patient measurement systems for generating respective alarm signals indicative of a patient's physiological condition, providing a centralized physiological monitoring station for receiving the alarm signals from the patient measurement systems, and receiving alarms indicative of respective physiological conditions of a plurality of patients at the centralized physiological monitoring station. The method also includes monitoring patterns in the alarms for each of the patients, establishing individualized historical alarm information for each of the patients responsive to monitored trends in the alarms, and determining an urgency of the respective alarms according to the individualized historical alarm information. The method further includes providing notifications to a user monitoring the respective physiological conditions at the centralized physiological monitoring station, wherein the notifications are prioritized according to the urgency of the alarms so as to reduce a workload on the user by limiting a need of the user to respond to less urgent alarms so that the user is able to concentrate on more urgent alarms.

In another example embodiment, the invention includes computer readable media containing program instructions for reducing a workload of a user monitoring respective physiological conditions of a plurality of patients at a centralized physiological monitoring station. The computer readable media includes a computer program code for providing a plurality of patient measurement systems for generating respective alarm signals indicative of a patient's physiological condition, a computer program code for providing a centralized physiological monitoring station for receiving the alarm signals from the patient measurement systems, and a computer program code for receiving alarms indicative of respective physiological conditions of a plurality of patients at the centralized physiological monitoring station. The computer readable media also includes a computer program code for monitoring patterns in the alarms for each of the patients and a computer program code for establishing individualized historical alarm information for each of the patients responsive to monitored trends in the alarms. The computer readable media also includes a computer program code for determining a urgency of the respective alarms according to the individualized historical alarm information and a computer program code for providing notifications to a user monitoring the respective physiological conditions at the centralized physiological monitoring station, wherein the notifications are prioritized according to the urgency of the alarms so as to reduce a workload on the user by limiting a need of the user to respond to less urgent alarms so that the user is able to concentrate on more urgent alarms.

In another example embodiment, the invention includes a system for reducing a workload of a user monitoring respective physiological conditions of a plurality of patients at a centralized physiological monitoring station. The system includes a plurality of patient measurement systems generating respective alarms indicative of a patient's physiological condition and a centralized physiological monitoring station receiving the alarms from the patient measurement systems. The system also includes a processing module in communication with the centralized physiological monitoring station configured for adaptively controlling a presentation of the alarms at the centralized physiological monitoring station responsive to at least one of patient history information, historical alarm information, electro-physiological data, quality of service data, and staff assignment information so as to reduce a workload on a user monitoring the presentation of the alarms and a display in communication with the adaptive processing module for displaying the presentation of the alarms to the user at the centralized physiological monitoring station.

### BRIEF DESCRIPTION OF THE DRAWINGS

FIG. 1 shows a block diagram of an example embodiment of the centralized monitoring system for reducing a workload of a tele-tech monitoring respective physiological conditions of a plurality of patients at a centralized physiological monitoring station.

FIG. 2 shows a schematic functional flow chart useful for understanding operation of the example system of FIG. 1.

FIG. 3 shows a flow chart useful for understanding a single-patient patient monitoring process of the example system of FIG. 1.

FIG. 4 shows a detailed flow chart useful for understanding a multi-patient system monitoring process of the example system of FIG. 1.

FIG. 5 shows an example graphical tele-tech interface (GUI) display format of the system of FIG. 1.

### DETAILED DESCRIPTION OF THE INVENTION

The inventors have recognized that conventional centralized patient monitoring systems may be prone to causing work overload conditions for tele-techs of the systems, such as tele-techs. For example, alarms generated by conventional monitoring equipment may not be immediately actionable by tele-techs monitoring the system, and may require the tele-tech to refer back to raw waveform data to verify the accuracy and consistency of the alarm. In addition, tele-techs may be required to manually note the status of actions in progress, which may take longer to complete than the persistence of the alarm itself. Another problem facing tele-techs is that staff assignments in hospitals tend to be highly dynamic, making it sometimes difficult for a tele-tech to locate an appropriate clinician to respond to an alarm. Furthermore, in wireless monitoring systems that include ambulatory patients, a higher number of artifacts, or false alarms, may be generated than for monitoring systems monitoring non-ambulatory patients. Although such false alarms generally are considered to have low priority, they may create a large distraction to the tele-tech monitoring many patients. Finally, typical centralized monitoring systems may not provide an indication of when or whether a requested intervention action has been performed, or whether it has been successful.

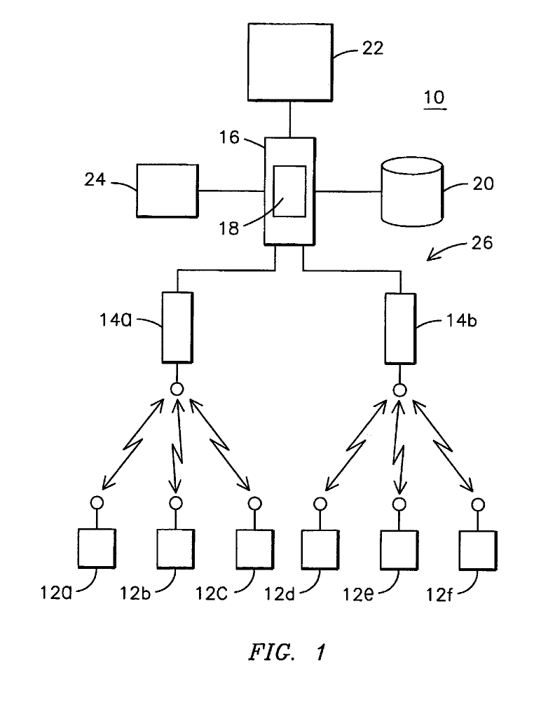

FIG. 1 shows a block diagram of an example embodiment of a centralized monitoring system 10 for reducing a workload of a tele-tech, such as a tele-tech monitoring respective physiological conditions of a plurality of patients at a centralized physiological monitoring station of the system. The system 10 may include a plurality of patient measurement systems 12a-12f generating respective signals indicative of a monitored patient's physiological condition. The patient measurement systems may include wireless transmitters that monitor various physiological parameters of a respective patient and transmit information to a network node 14a, 14b, such as via a wireless connection. Information available at the network node may include patient admission or discharge information, alarm configuration information, transient waveform information, and alarms generated for a patient. In an example embodiment, the network node may serve a ward or floor of a hospital. Each of the nodes may forward signals received from the respective measurement systems 12a-12f to a centralized physiological monitoring station 16, such as via a dedicated digital network 26.

The centralized physiological monitoring station 16 may provide information, such as visual and auditory indicia, on display 22. The centralized physiological monitoring station 16 may be in communication with one or more databases 20, such as a hospital information database (HIS) capable of supplying additional information such as admissions data, patient medical history, or laboratory test results. The centralized physiological monitoring station 16 may also be in communication with a medical personnel notification system (PNS) 24 that may in turn include paging, digital voice (voice-over IP), or telephony capability, an auditory alarm system, and/or a personnel tracking system (e.g., badges, RFID) and/or security system.

In an aspect of the invention, the centralized monitoring system 10 is configured to reduce the workload of centralized monitoring staff, such as tele-techs, and, by extension, reduce workloads of primary clinicians responsible for care and treatment of inpatients at a medical facility. Accordingly, the centralized monitoring system 10 may include a processor 18 in communication with the centralized physiological monitoring station configured to monitor patterns in alarms for each of the patients and to establish individualized historical alarm information for each of the patients responsive to monitored patterns in the alarms. The historical alarm information may be stored in a database 20. The processor 18 may also be configured to determine an urgency of the respective alarms according to the individualized historical alarm information. In addition, the processor 18 may be configured be to provide notifications to a user, such as a tele-tech, monitoring the respective physiological conditions at the centralized physiological monitoring station 10. The notifications may be prioritized according to the urgency of the alarms so as to reduce a workload on the tele-tech. By providing notifications prioritized according to urgency, a need of the tele-tech to respond to less urgent alarms may be reduced so that the tele-tech is able to concentrate on more urgent alarms.

In an example embodiment, the processor 18 may be configured to identify ones of the alarms that do not require a response by the tele-tech and to ignore these alarms. The processor 18 may also be configured to establish historical false alarm information based on ones of the alarms that do not require a response, and to store this information in a database, such as database 20. In other example embodiments, the processor 18 may be configured to determine the urgency of the respective alarms according to predetermined guidelines and/or an availability of responders to the respective alarms.

The processor 18 may further be configured to determine whether a tele-tech has responded to the notifications and to modify priorities of notifications when the tele-tech has not responded within a predetermined time. In another example embodiment, the processor 18 may be configured to establish respective tasks to be performed responsive to the notifications, and to determine when the respective tasks have been completed. The processor may also be configured to establish historical task information responsive to the respective tasks that have been completed. This information may be stored in a database, such as database 20, and accessed to determine the urgency of the respective alarms according to the historical task information stored therein. The processor 18 may also be configured to access patient admission data, for example, stored in database 20. The system 10 may further include a display in communication with the processor 18 for providing the notification to, i.e., alerting of, the tele-tech, such as by providing visual and/or auditory indicia corresponding to a priority of the notification.

Accordingly, the operation of the system 10 includes screening and verifying alarms received from patient measurement systems, classifying alarms by severity, and determining what form of notification is appropriate for a tele-tech and/or for clinicians on a ward. Workflow tasks may be initiated for alarms that require the involvement of medical personnel, and such tasks may include the incorporation of paging, telecommunications, or remote display devices in proximity to medical personnel. Other tasks may also be initiated and/or remotely displayed for other medical personnel, such as ward administrative personnel, tele-runners (who change batteries), and/or aides, when patients being monitored wirelessly are out-of-range of a receiving node. Tasks may be associated with a specific patient, and may include display indicia corresponding to an urgency of the task. Completed tasks may be logged into a case base that may be used, along with historical data from each patient, to automatically adjust alarm configuration suitable to a condition of the patient. This may involve the adjustment of alarm priorities and/or limits (for example, using machine learning) to adapt alarms to the patient's current state or medical condition. The information stored in the case base may also be used to augment historical data, and to support quality-of-service monitoring, auditing, and staffing system information.

The system 10 may be configurable for different hospital workflow practices, staffing policies, patient management protocols, etc. The system 10 may automatically provide adjustments suitable for staffing shortages periods of high workload, such as power outages or local emergencies), or other hospital-related circumstances. The system 10 may further include automatic physician notification outside the hospital environment.

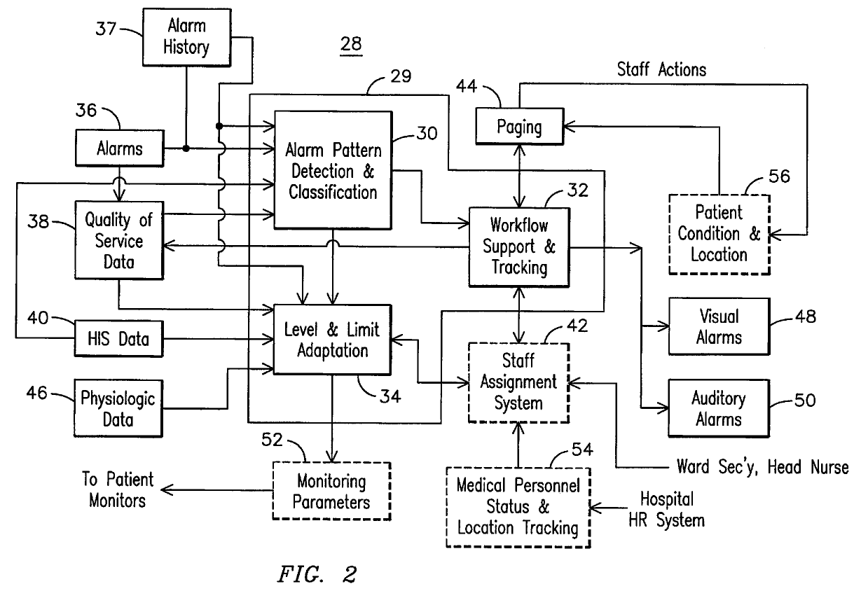

FIG. 2 shows a schematic functional flow chart 28 useful for understanding operation of the example system 10 of FIG. 1. The flow chart 28 includes an adaptive processing block 29 that may include three primary processing blocks: alarm pattern detection and classification block 30, workflow support and tracking block 32, and level and limit adaptation block 34 for adaptively controlling presentation of alarms. Alarm pattern detection and classification block 30 may include alarm filtering by screening out false alarms, for example, based on knowledge that a tele-tech would normally use; identifying patterns to detect root causes of false alarms; providing suggested measures for clearing alarms; and classifying alarm patterns to infer patient physiological state for health trend prediction. Accordingly, alarm pattern detection and classification block 30 may serve to filter out repeated alarms in a series or pattern of alarms generated by a single event. Inputs to the alarm pattern detection and classification block 30 may include alarms 36 generated by a patient monitor; HIS data 40, such as data derived from patient admission data; statistics data from a quality-of-service data 38, historical alarm information from an alarm history 37; and/or other sources of a patient's clinical information. The inputs may be processed in the alarm pattern detection and classification block 30 to determine if an alarm is an erroneous, or false alarm, and to provide suggested measures to implement for responding to a clinically-relevant alarm. Accordingly, the alarm pattern detection and classification block 30 may perform intelligent alarm reduction to reduce a workload of a tele-tech, such as by automatically identifying clinically-relevant alarms and determining a severity of alarms. The alarm pattern detection and classification block 30 may also provide a task list of prioritized alarms to be cleared with suggested measures to take for each alarm, and a status list of current open/in-progress alarms to reduce occurrence of missed alarms.

Alarms may be classified using a variety of known techniques. In an embodiment, a technique incorporating unsupervised learning, such as the k-means clustering algorithm, or semi-supervised learning may be used. Support vector machine algorithms for clinical alarm pattern detection and classification may also be used to classify alarms. For example, given a large unlabeled dataset, such as a dataset of sequential clinical alarms, a k-mean clustering algorithm may be used to select a small, e.g., 5% to 10%, representative sample for alarm pattern detection. In another example embodiment, a combined labeled-unlabeled dataset may be classified using a semi-supervised support vector machine (SVM). In another example embodiment, a growing self-organizing map (GSOM) and an SVM machine technique may be used for alarm pattern detection and classification.

Workflow support and tracking block 32 may prioritize alarms and provide suggested measures for responding to the alarms, perform job scheduling and resource optimization, and dispatch clinicians for alarm servicing. The workflow support and tracking block 32 may also manage current active or in-progress alarms and update an individual's alarms status. For example, the workflow support and tracking block 32 may be configured for determining whether alarms have had a respond and reprioritizing ones of the alarms that have not had a respond. To perform the above procedures, the workflow support and tracking block 32 may receive information from the alarm pattern detection and classification block 30, information from a staff assignment system 42, feedback from a paging system 44, for example to verify that tasks have acted upon, and/or from quality-of-service data 38. The workflow support and tracking block 32 may generate prioritized visual and/or auditory alarms based on the information provided.

The level and limit adaptation block 34 may adaptively change parameter settings 52 of patient measurement systems based on an individual patient's alarm patterns. The level and limit adaptation block 34 may receive electro-physiological data 46, information from the alarm pattern detection and classification block 30, information from a staff assignment system 42 (derived, for example, from medical personnel status and location tracking 54), feedback from a paging system 44 to verify that tasks have acted upon, (responsive, for example, to patient condition and location 56), historical alarm information from an alarm history 37; and/or information from quality metrics 38. Alarms limits and levels may be determined using classification methods such as a known boosting decision tree technique and/or a known neural network technique.

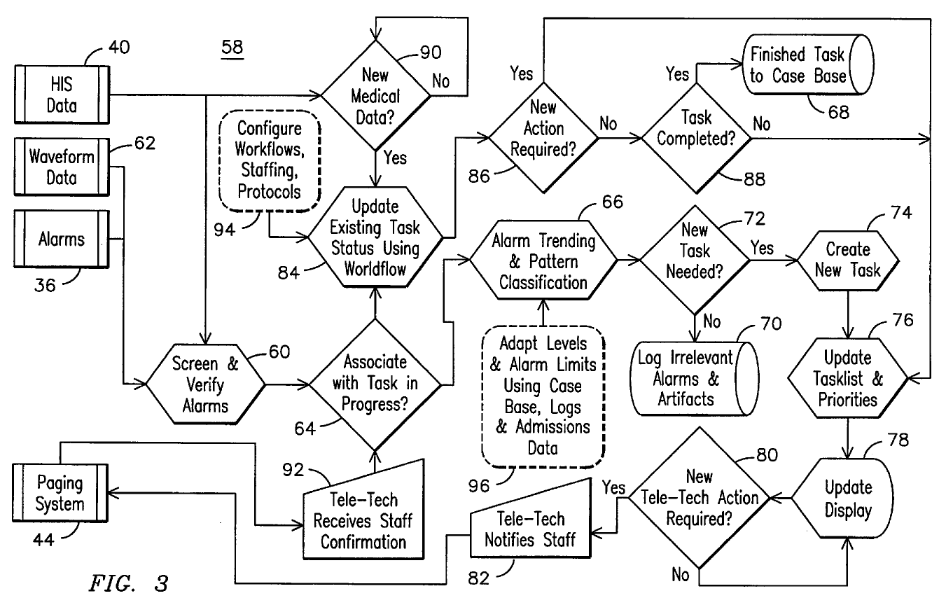

FIG. 3 shows a flow chart useful for understanding a single patient monitoring process of the example system of FIG. 1. Alarms 36 and waveform data 62 received from patient measurement systems may be screened and verified 60, for example, based on HIS data 40, such as hospital admission information. Alarms may then be associated with tasks 64 that have previously been established, such as tasks that have been established for other alarms previously received. The alarm may then be classified 66 based on classification information 96 that may be provided by the level and limit adaptation block 34 of FIG. 2. The classification information 96 may be derived from a case base 68, a log 70, such as a log of irrelevant and artifact alarms corresponding to tasks previously performed, and/or HIS data 40, such as admission data. When a new task is need 72, a new task may be created and a task list updated 76. Based on the updated task list, the display may be updated 78. If new tele-tech action is required 80, tele-tech notifies the appropriate medical staff, such a clinician, for example, via paging system 44. When the tele-tech receives staff confirmation 92 that the task has been responded to, processing returns to associating alarms with tasks 64. In conjunction with alarm classification and notification, task status of assigned tasks may be either manually or automatically updated 84 using workflow data based on staffing and protocol information 94 and new medical information 90. When a new action is required 86, the task list may be automatically updated 76. Otherwise, a determination is made whether a task has been completed 88. If a task has been completed, information about the task may be stored in the case base 68. If the task has not been completed, the task list is updated 76 accordingly.

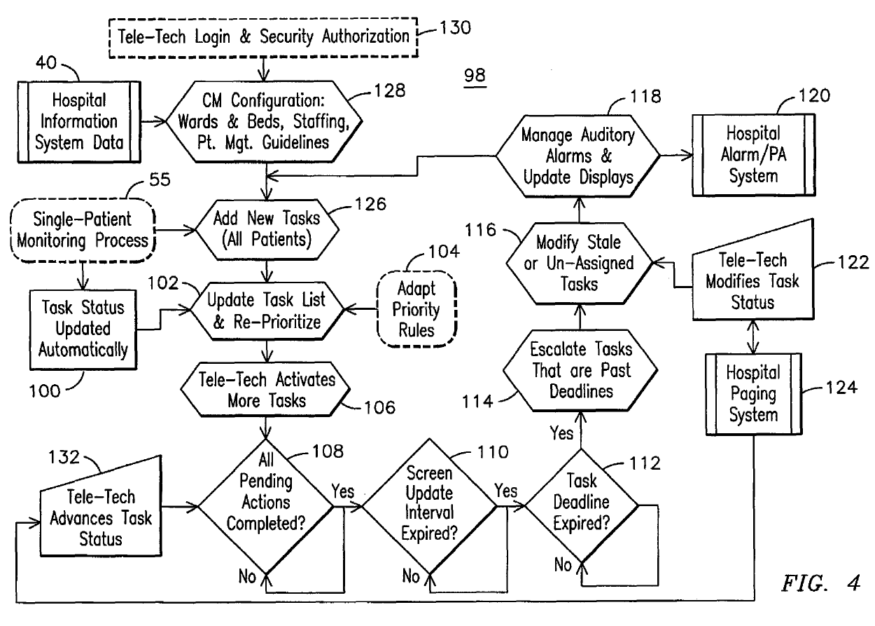

FIG. 4 shows a flow chart useful for understanding a multi-patient monitoring process of the example system 10 of FIG. 1. The flow chart 98 may incorporate the patient monitoring flow 55 depicted in the flow chart 54 of FIG. 3. The process depicted in flow chart 98 may include a facility for logging in a user 130, such as a tele-tech. The process may be configured according to beds, wards, staffing, and/or patient management guidelines 128 that may be available in HIS data 40. The process may include adding new tasks for all patients being monitored 126, automatically updating task statuses 100, and updating and re-prioritizing a task list 102, for example, according to adaptive priority rules 104. After a tele-tech activates more tasks 106, or advances task status 132, the process may include determining whether any pending actions have been completed 108. If pending actions have been completed, processing continues with determining whether a screen update interval, such as an update interval of 1 to 3 seconds, has expired 110. If a screen update interval has expired, processing continues with determining whether any task deadlines have expired 112. If any task deadlines have expired, these tasks may be escalated 114, such as by increasing their priority, level, for example, indicative of an urgency of the task. Next, stale or unassigned tasks may be modified 116 responsive to a tele-tech's input modifications 122 based, for example, on hospital paging system 44 information, such as a special manual inquiry concerning whether the action is completed. The system then updates a display 118 according to new and/or modified task information and may include notifying a hospital alarm or PA system 120. Processing may then return to adding new tasks 126. Typically, the processing loop from block 126 to block 118 progresses at an event-driven screen or task update rate, usually every few seconds.

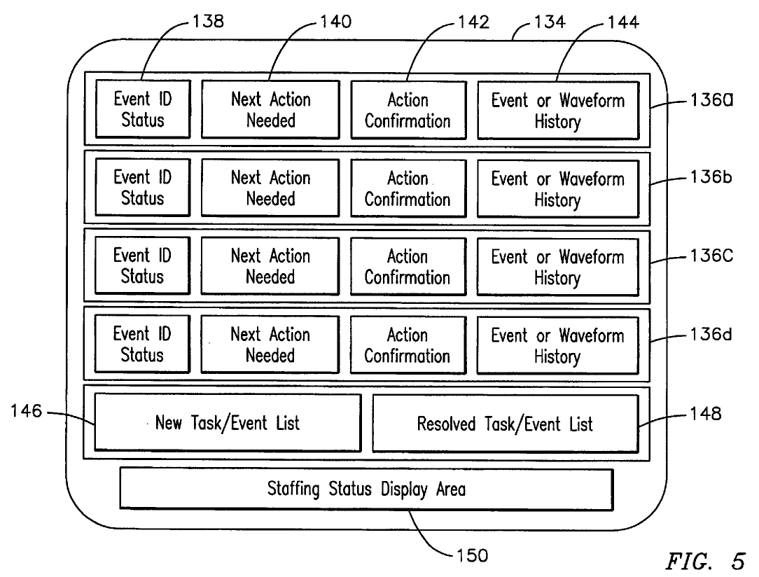

FIG. 5 shows an example graphical user interface (GUI) display format 134 of the system of FIG. 1. The display format 134 may include visual indicia for maintaining and displaying task information responsive to processed alarms. The display format 134 may also include a plurality of active task indicia 136a . . . 136d indicative of active tasks, and may be arranged and or presented so as to indicate a priority of tasks according to a determined urgency. Each task indicia may include an event ID status indication 138 for displaying a type of task, a next action needed indication 140 for displaying the next action needed to be taken, an action confirmation indication 142 that may be activated to confirm action has been taken, and an event or waveform history indication 144 that may include historical information related to the event ID and/or historical waveform information. The display format 134 may also include new task list indicia 146 indicative of tasks waiting to be activated, and a resolved task list indicia 148 indicative of recently resolved tasks. The display format 134 may also include a staffing status display indicia 150 for showing availability of responders, such as clinicians on respective wards.

Based on the foregoing specification, the invention may be implemented using computer programming or engineering techniques including computer software, firmware, hardware or any combination or subset thereof, wherein the technical effect is reducing a workload of a tele-tech monitoring physiological conditions of a plurality of patients at a centralized physiological monitoring station. Any such resulting program, having computer-readable code means, may be embodied or provided within one or more computer-readable media, thereby making a computer program product, i.e., an article of manufacture, according to the invention. The computer readable media may be, for instance, a fixed (hard) drive, diskette, optical disk, magnetic tape, semiconductor memory such as read-only memory (ROM), etc., or any transmitting/receiving medium such as the Internet or other communication network or link. The article of manufacture containing the computer code may be made and/or used by executing the code directly from one medium, by copying the code from one medium to another medium, or by transmitting the code over a network.

One skilled in the art of computer science will easily be able to combine the software created as described with appropriate general purpose or special purpose computer hardware, such as a microprocessor, to create a computer system or computer sub-system embodying the method of the invention. An apparatus for making, using or selling the invention may be one or more processing systems including, but not limited to, a central processing unit (CPU), memory, storage devices, communication links and devices, servers, I/O devices, or any sub-components of one or more processing systems, including software, firmware, hardware or any combination or subset thereof, which embody the invention.

While certain embodiments of the present invention have been shown and described herein, such embodiments are provided by way of example only. Numerous variations, changes and substitutions will occur to those of skill in the art without departing from the invention herein. Accordingly, it is intended that the invention be limited only by the spirit and scope of the appended claims.

What is claimed is:

1. A system for reducing a workload of a user monitoring respective physiological conditions of a plurality of patients at a centralized physiological monitoring station comprising:
a plurality of patient measurement systems generating respective alarms indicative of a patient's physiological condition;
a centralized physiological monitoring station receiving the alarms from the patient measurement systems;
a processor in communication with the centralized physiological monitoring station configured to monitor patterns in the alarms for each of the patients, to establish individualized historical alarm information for each of the patients responsive to monitored patterns in the alarms, to determine an urgency of the respective alarms according to the individualized historical alarm information, and to provide notifications to a user monitoring the respective physiological conditions at the centralized physiological monitoring station, wherein the notifications are prioritized according to the urgency of the alarms so as to reduce a workload on the user by limiting a need of the user to respond to less urgent alarms so that the user is able to concentrate on more urgent alarms; and
a display in communication with the processor for displaying the notifications to the user.

2. The system of claim 1, wherein the processor is further configured to identify ones of the alarms that do not require a response by the user.

3. The system of claim 2, wherein the processor is further configured to ignore the ones of the alarms that do not require a response by the user.

4. The system of claim 3, wherein the processor is further configured to establish historical false alarm information based on the ones of the alarms that do not require a response.

5. The system of claim 4, further comprising a database accessible by the processor for storing historical alarm information, wherein the processor is further configured to store the historical false alarm information in the database.

6. The system of claim 1, wherein the processor is further configured to determine the urgency of the respective alarms according to predetermined guidelines.

7. The system of claim 1, wherein the processor is further configured to determine whether a user has responded to the notifications.

8. The system of claim 1, wherein the processor is further configured to modify priorities of the notifications when the user has not responded to the notifications within a predetermined time.

9. The system of claim 1, wherein the processor is further configured to establish respective tasks to be performed responsive to the notifications.

10. The system of claim 9, wherein the processor is further configured to determine when the respective tasks have been completed.

11. The system of claim 10, wherein the processor is further configured to establish historical task information responsive to the respective tasks that have been completed.

12. The system of claim 11, further comprising a database accessible by the processor for storing historical task information, wherein the processor is further configured to determine the urgency of the respective alarms according to the historical task information stored in the database.

13. The system of claim 1, wherein the processor is further configured to determine the urgency of the respective alarms according to an availability of responders to the respective alarms.

14. The system of claim 1, further comprising a database accessible by the processor for storing patient admission data, wherein the processor is further configured to determine the urgency of the respective alarms according to the patient admission data stored in the database.

15. A method for reducing a workload of a user monitoring respective physiological conditions of a plurality of patients at a centralized physiological monitoring station comprising:
providing a plurality of patient measurement systems for generating respective alarm signals indicative of a patient's physiological condition;
providing a centralized physiological monitoring station for receiving the alarm signals from the patient measurement systems;
receiving alarms indicative of respective physiological conditions of a plurality of patients at the centralized physiological monitoring station;
monitoring patterns in the alarms for each of the patients;
establishing individualized historical alarm information for each of the patients responsive to monitored trends in the alarms;
determining an urgency of the respective alarms according to the individualized historical alarm information; and
providing notifications to a user monitoring the respective physiological conditions at the centralized physiological monitoring station, wherein the notifications are prioritized according to the urgency of the alarms so as to reduce a workload on the user by limiting a need of the user to respond to less urgent alarms so that the user is able to concentrate on more urgent alarms.

16. The method of claim 15, further comprising identifying ones of the alarms that do not require a response by the user.

17. The method of claim 16, further comprising ignoring the ones of the alarms that do not require a response by the user.

18. The method of claim 16, further comprising establishing historical false alarm information based on the ones of the alarms that do not require a response.

20. The method of claim 15, further comprising determining the urgency of the respective alarms according to predetermined guidelines.

21. The method of claim 15, further comprising determining whether a user has responded to the notifications.

22. The method of claim 21, further comprising, when the user has not responded to the notifications within a predetermined time, modifying priorities of the notifications to which the user has not responded.

23. The method of claim 15, further comprising establishing respective tasks to be performed responsive to the notifications.

24. The method of claim 23, further comprising determining when the respective tasks have been completed.

25. The method of claim 24, further comprising establishing historical task information responsive to the respective tasks that have been completed.

26. The method of claim 25, further comprising determining the urgency of the respective alarms according to the historical task information.

27. The method of claim 15, further comprising determining the urgency of the respective alarms according to an availability of responders to the alarms.

28. The method of claim 15, further comprising determining the urgency of the respective alarms according to patient admissions data.

29. Computer readable media containing program instructions for reducing a workload of a user monitoring respective physiological conditions of a plurality of patients at a centralized physiological monitoring station, the computer readable media comprising:
a computer program code for providing a plurality of patient measurement systems for generating respective alarm signals indicative of a patient's physiological condition;
a computer program code for providing a centralized physiological monitoring station for receiving the alarm signals from the patient measurement systems;
a computer program code for receiving alarms indicative of respective physiological conditions of a plurality of patients at the centralized physiological monitoring station;
a computer program code for monitoring patterns in the alarms for each of the patients;
a computer program code for establishing individualized historical alarm information for each of the patients responsive to monitored trends in the alarms;
a computer program code for determining a urgency of the respective alarms according to the individualized historical alarm information; and
a computer program code for providing notifications to a user monitoring the respective physiological conditions at the centralized physiological monitoring station, wherein the notifications are prioritized according to the urgency of the alarms so as to reduce a workload on the user by limiting a need of the user to respond to less urgent alarms so that the user is able to concentrate on more urgent alarms.

30. A system for reducing a workload of a user monitoring respective physiological conditions of a plurality of patients at a centralized physiological monitoring station comprising:
a plurality of patient measurement systems generating respective alarms indicative of a patient's physiological condition;
a centralized physiological monitoring station receiving the alarms from the patient measurement systems;
a processing module in communication with the centralized physiological monitoring station configured for adaptively controlling a presentation of the alarms at the centralized physiological monitoring station responsive to at least one of patient history information, historical alarm information, electro-physiological data, quality of service data, and staff assignment information so as to reduce a workload on a user monitoring the presentation of the alarms; and
a display in communication with the adaptive processing module for displaying the presentation of the alarms to the user at the centralized physiological monitoring station.

31. The system of claim 30, wherein the processing module further comprises an alarm classification module configured for detecting alarm patterns in the alarms and classifying the alarms according to the detected alarm patterns, so that a number of the alarms presented to a user may be reduced compared to presenting all the alarms generating the pattern.

32. The system of claim 31, wherein the alarm classification module is further configured for identifying alarm patterns indicative of false alarms.

33. The system of claim 32, wherein the alarm classification module is further configured for identifying causes of the detected alarm patterns.

34. The system of claim 30, wherein the alarm classification module is further configured for predicting a health trend of a patient responsive to the detected alarm patterns.

35. The system of claim 30, wherein the processing module further comprises a workflow tracking module in communication with the alarm classification module configured for prioritizing the alarms and determining appropriate responses to the alarms.

36. The system of claim 35, wherein the workflow tracking module is further configured for communicating with a staff assignment system to retrieve staffing information and for determining staff available to provide the appropriate responses to the alarms based on at least the staffing information.

37. The system of claim 36, wherein the workflow tracking module is further configured for communicating with a paging system for paging at least one of the staff available to provide the appropriate responses to the alarms.

38. The system of claim 35, wherein the workflow tracking module is further configured for determining whether the alarms have had a response and modifying a priority ones of the alarms that have not had a response.

39. The system of claim 35, wherein the workflow tracking module is further configured for generating at least one of a visual and audio presentation of the alarms.

40. The system of claim 30, wherein the processing module further comprises an alarm level module configured for adaptively modifying settings of the patient measurement systems.
